(19) United States
(12) Patent Application Publication
(10) Pub. No.: US 2013/0131648 A1
(43) Pub. Date: May 23, 2013

(54) SURGICAL HOOK

(71) Applicant: SOUHEIL FUAD HADDAD, Bloomington, IN (US)

(72) Inventor: SOUHEIL FUAD HADDAD, Bloomington, IN (US)

(21) Appl. No.: 13/301,017

(22) Filed: Nov. 21, 2011

(51) Int. Cl. A61B 17/00

(52) U.S. Cl. 606/1

(57) ABSTRACT

A surgical tool to safely penetrate the posterior longitudinal ligaments and prevent damage to vital intervening tissues near the spine. The surgical tool includes a handle, a shaft secured to the handle, and a hook secured to the shaft. The hook extends from the shaft and includes a blunt outside edge, a sharp inside edge, and a blunt tip.

### BACKGROUND OF THE INVENTION

The present invention generally relates to a surgical tool. More particularly, the present invention relates to a tool to open posterior longitudinal ligaments in spine surgery.

In minimally invasive spine surgical procedures, it is important to minimize the trauma to patients and damage to vital intervening tissues near the spine. Several surgical tools have been developed to be used in spinal surgery in order to prevent the damage to vital intervening tissues near the spine. Unfortunately, the known surgical tools require the surgeon to physically move or move the surgical tool in order to have a better visualization of the tip of the surgical tool relative to a spine working point. In addition, the known surgical tools are not strong enough to safely penetrate the posterior longitudinal ligaments without damaging the adjacent tissues. Moreover often a sharp knife is used to open the posterior longitudinal ligament. The blade is pointing toward the ligament and the spinal cord. If the blade is inadvertently pushed too far, it will open the dura, covering of the spinal cord and may even injure the spinal cord resulting in possible spinal cord injury and paralysis.

As can be seen, there is a need for a surgical tool for spine surgery that safely penetrates the posterior longitudinal ligaments and prevents damage to vital intervening tissues near the spine. In addition, there is a need for a surgical tool that allows the surgeon to use the surgical tool without having to change position to visualize the spine working point.

### SUMMARY OF THE INVENTION

In one aspect of the present invention, a surgical tool includes a handle, a shaft secured to the handle, a hook secured to the shaft, and the hook extends from the shaft and includes a blunt outside edge, a sharp inside edge, and a blunt tip.

In another aspect of the present invention, a surgical tool includes a handle; a z-shaped shaft secured to the handle; a hook secured to the shaft; the hook extends from the shaft and includes a blunt outside edge, a sharp inside edge, and a blunt tip; the hook is perpendicular and offset from the handle; and the shaft is parallel and offset from the handle.

These and other features, aspects and advantages of the present invention will become better understood with reference to the following drawings, description and claims.

### BRIEF DESCRIPTION OF THE DRAWINGS

FIG. 1 is a perspective front view of a surgical tool according to an exemplary embodiment of the present invention;

FIG. 2 is another perspective front view of the surgical tool of FIG. 1 taken from a different angle;

FIG. 3 is a side view of the surgical tool of FIG. 1;

FIG. 4 is a top view of the surgical tool of FIG. 1 shown from line 4-4 in FIG. 3;

FIG. 5 is a side view of the surgical tool of FIG. 1 shown from line 5-5 in FIG. 4; and

FIG. 6 is a cross-section view of the surgical tool of FIG. 1 shown from line 6-6 in FIG. 1.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, embodiments of the present invention generally provide a surgical tool to open the posterior longitudinal ligament (PLL) in spine surgery.

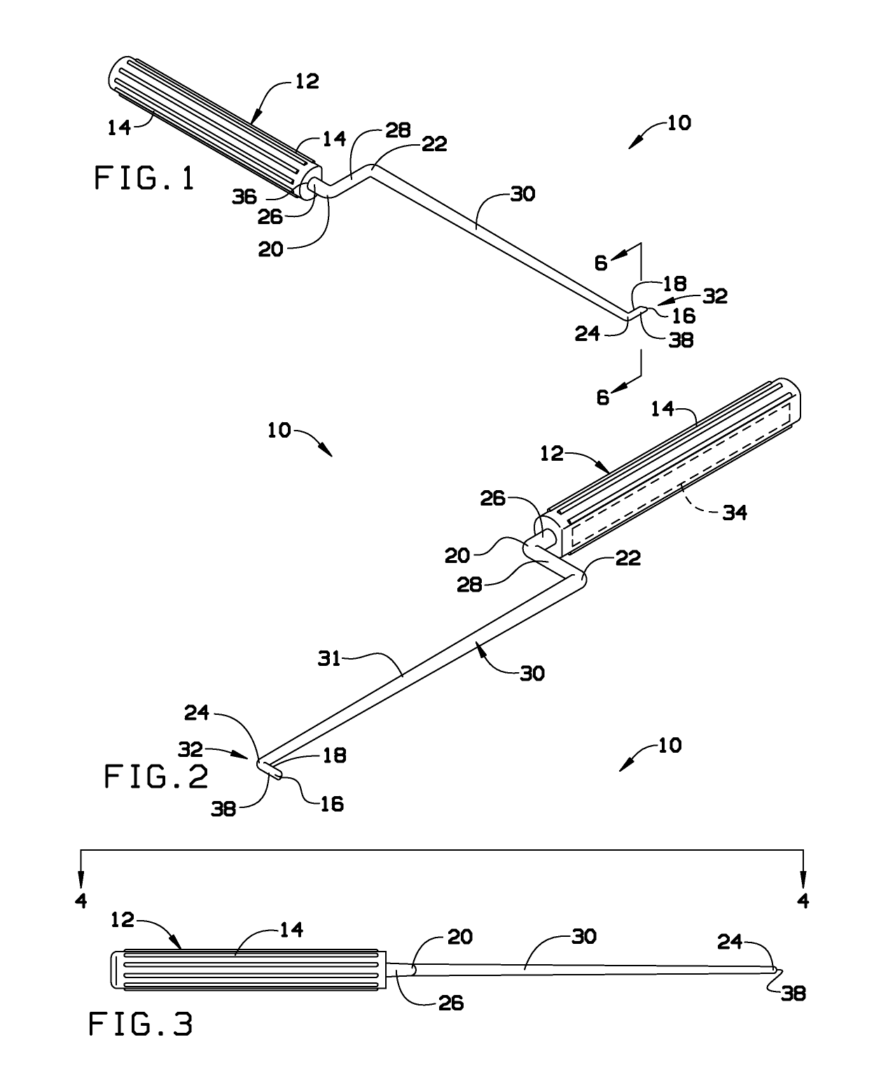

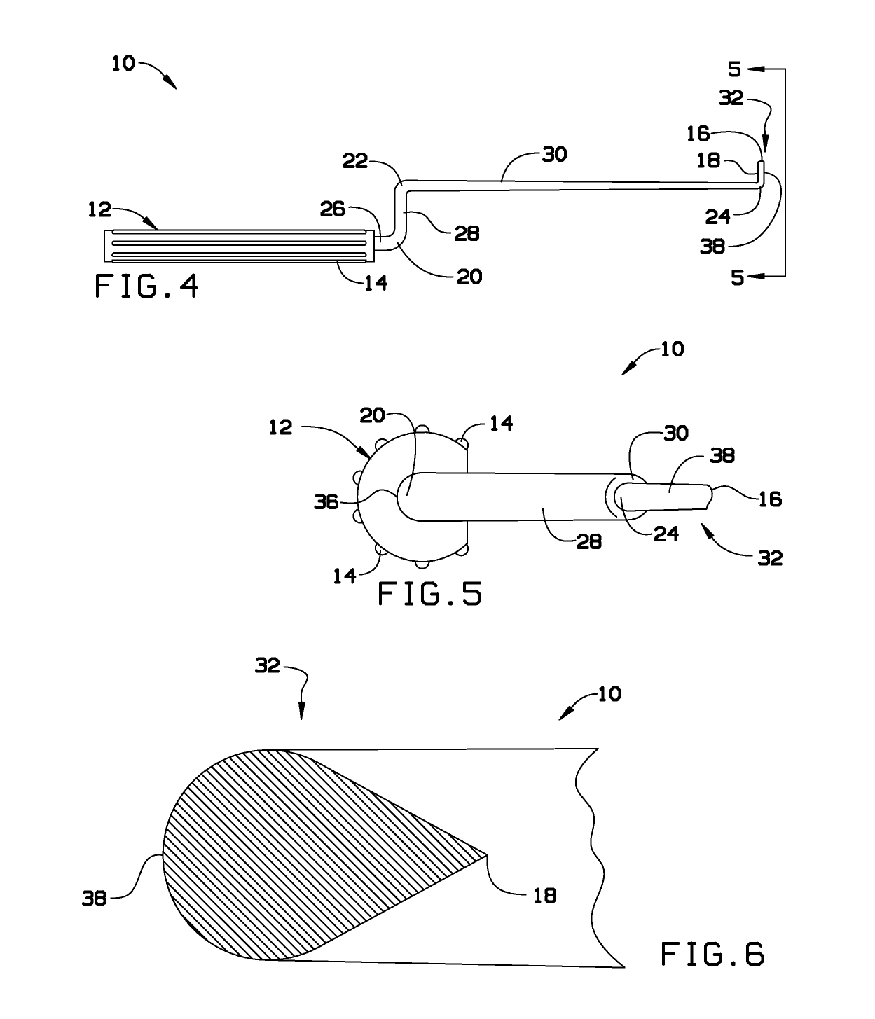

FIGS. 1-6 show a surgical tool 10 according to an exemplary embodiment of the present invention. The surgical tool 10 may be used to safely penetrate the posterior longitudinal ligaments and prevent damage to vital intervening tissues near the spine. In addition, the surgical tool 10 may allow a surgeon (not shown) to use the surgical tool 10 without having to change position to visualize the spine working point (not shown).

The surgical tool 10 may include a handle 12, a shaft 30 secured to the handle 12, and a hook 32 secured to the shaft 30.

The handle 12 may be made of a sturdy material, for example, metal, plastic, polymer, composite, or wood. The size of the handle 12 may depend on the surgeon preferences. The handle 12 may have a shape that facilitates the gripping of the surgical tool 10, for example, a cylindrical, rectangular, triangular, square, pentagonal, or hexagonal.

Rib grips 14 may be formed around the handle 12 to secure the holding of the surgical tool 10.

A panel 34 may be secured to the handle 12. The panel 34 may include advertising information, for example, logos, phrases, figures, letters, or phone numbers.

The shaft 30 may be secured to the handle 12 by inserting the shaft into a hole 36 on the handle. The shaft 30 may be secured to the hole 36 by using a standard attaching method, for example, molding, welding, gluing, threading, or fasteners. The shaft 30 may be made of a medical grade material that is sufficiently rigid to withstand the force needed to properly align and penetrate the surgical tool 10 into the posterior longitudinal ligament without the risk of breaking. The shaft 30 may be made of, for example, medical grade stainless steel or titanium.

The length, shape, and thickness of the shaft 30 may depend on the type of surgery. The shaft 30 may be a straight shaft or an offset shaft.

In some embodiments, the shaft 30 may have a z-shape. In this embodiment, the shaft 30 may be divided into a first section 26, a second section 28, and a third section 31. The first section 26 may be connected to the handle 12. The second section 28 may perpendicularly extend from the first section 26 forming a first bending point 20. The third section 31 may perpendicularly extend from the second section 26 in a direction opposite to the first section and may form a second bending point 22. The bending points 20, 22 may produce the third section 31 to be substantially parallel and offset from the handle 12 and the first section 26. The first section 26 may be offset from the handle 12, for example, approximately 1 cm. The second section 28 may be offset from the handle 12, for example, approximately between 2 and 3 cm. The third section 31 may be offset from the handle 12, for example, approximately between 10 and 15 cm.

The size of the shaft 30 may gradually taper from the first section 26 to the third section 31; thus, the third section may be small enough to enter the disk space (not shown) and ligaments (not shown) of the patient (not shown). In addition, the tapering may help with the visualization of the hook 32.

The hook 32 may perpendicularly extend from the third section 31 of the shaft 30 forming a third bending point 24. The bending point 24 may produce the hook 32 to be substantially perpendicular and offset from the handle 12. The hook 32 may include a blunt outside edge 38, a sharp inside edge 18, and a blunt tip 16. The sharp inside edge 18 may extend from the third section 31, for example, approximately between 2 and 15 mm.

The hook 32 may be offset from the handle 12, for example, between 3 and 6 mm. The offset between the handle 12 and the hook 32 may provide the user (not shown) with the required space to placed the surgical tool 10 in the disc space and maneuver it into the ligament.

The blunt outside edge 38 and the blunt tip 16 may allow the surgeon to penetrate the posterior longitudinal ligament during spine surgery without damaging the adjacent tissues.

The offset arrangement between the shaft 30, the handle 12, and the hook 32 may allow the surgeon to use the surgical tool 10 without having to change position to visualize the spine working point.

The surgeon (not shown) may wield the surgical tool 10 by holding the handle 12. The surgeon may penetrate the hook 32 in the intervertebral space (not shown) of the spine (not shown). Then, the surgeon may push the blunt tip 16 of the hook 32 through the posterior longitudinal ligament either parallel or at an angle with the disk space. The surgeon may pull up or slide the sharp inside edge 18 of the hook 32 to open the posterior longitudinal ligament.

The surgical tool 10 may be used in any field where the surgical objective is to cut tissue while protecting any underlying tissue.

It should be understood, of course, that the foregoing relates to exemplary embodiments of the invention and that modifications may be made without departing from the spirit and scope of the invention as set forth in the following claims.

1. A surgical tool comprising:
a handle;
a shaft directly secured to the handle; and
a hook secured to the shaft and extending from the shaft and including a blunt outside edge, a sharp inside edge, and a blunt tip.

2. The surgical tool according to claim 1, wherein the shaft is constructed from medical grade stainless steel or titanium.

3. The surgical tool according to claim 1, wherein the shaft has one end connected to the handle and another end connected to the hook, and tapers from the handle to the hook.

4. The surgical tool according to claim 1, wherein the shaft is an offset shaft.

5. The surgical tool according to claim 1, further including a panel secured to the handle for displaying advertising information.

6. The surgical tool according to claim 1, further including a plurality of rib grips secured to the handle.

7. A surgical tool comprising:
a handle;
a z-shaped shaft secured to the handle having a first section extending away from the handle, a second section transversely extending from the first section, and a third section extending away from the second section at a direction opposite to the direction the first section extends away from the second section to form a z-shaped cross section of the z-shaped shaft, the third section extending substantially parallel to the first section and substantially offset from and parallel to an axis extending through the handle; and
a hook secured to and extending from the z-shaped shaft, the hook including a blunt outside edge, a sharp inside edge, and a blunt tip, the hook extending away from the shaft in a direction perpendicular to the handle and offset from an axis extending through the handle.

8. The surgical tool according to claim 7, wherein the shaft is constructed from medical grade stainless steel or titanium.

9. The surgical tool according to claim 7, wherein the shaft has one end connected to the shaft and another end connected to the hook, and tapers from the handle to the hook.

10. The surgical tool according to claim 7, wherein the second section of the z-shaped shaft is perpendicularly extended away from an axis extending through the first section.

11. The surgical tool according to claim 7, wherein the third section of the z-shaped shaft is perpendicularly extended away from the second section.

12. The surgical tool according to claim 7, wherein the hook is perpendicularly extended from the third section of the z-shaped shaft.

13. A surgical tool comprising:
a handle;
a z-shaped shaft directly secured to the handle; and
a hook secured to and extending from the z-shaped shaft, the hook including a blunt outside edge, a sharp inside edge, and a blunt tip, the hook extending away from the shaft in a direction perpendicular to an axis extending through the handle and offset from the axis extending through the handle.

14. The surgical tool according to claim 13, wherein the shaft is constructed from medical grade stainless steel or titanium.

15. The surgical tool according to claim 13, wherein the shaft has one end connected to the handle and another end connected to the hook, and is tapers from the handle to the hook.
